(19) United States
(12) Patent Application Publication
(10) Pub. No.: US 2023/0158149 A1
(43) Pub. Date: May 25, 2023

(54) Wafer-Scale Protein Patterning of Hydrogel Devices

(71) Applicant: The Regents of the University of California, Oakland, CA (US)

(72) Inventors: Beth L. Pruitt, Santa Barbara, CA (US); Erica A. Castillo, Santa Barbara, CA (US); Anna Kim, Santa Barbara, CA (US); Gabriela Villalpando Torres, Santa Barbara, CA (US); Kerry V. Lane, Santa Barbara, CA (US)

(21) Appl. No.: 17/972,306

(22) Filed: Oct. 24, 2022

Related U.S. Application Data
(60) Provisional application No. 63/274,309, filed on Nov. 1, 2021.

(51) Int. Cl. A61K 47/42; A61L 27/52; B82Y 40/00

(52) U.S. Cl. CPC A61K 47/42; A61L 27/52; B82Y 40/00

(57) ABSTRACT

A method for performing protein patterning is provided. In particular, the method comprises generating a photoresist pattern on a wafer using photolithography; dicing the photoresist patterned wafer into wafer chips; and performing lift-off protein patterning on the photoresist patterned wafer chips. This manufacturing technique scales up the photolithography stage of the lift-off fabrication workflow to generate high quantities of shelf-stable photoresist patterned glass substrates, which can be used to incorporate a protein pattern onto the surface of a hydrogel.

### CROSS-REFERENCE TO RELATED APPLICATIONS

This application claims the benefit of U.S. Provisional Patent Application No. 63/274,309, filed Nov. 1, 2021, which application is incorporated herein by reference in its entirety.

### STATEMENT REGARDING FEDERALLY SPONSORED RESEARCH OR DEVELOPMENT

This invention was made with government support under Grant No. GM131981 and Grant No. TR002588 awarded by the National Institutes of Health, and Grant No. 1834760 awarded by the National Science Foundation. The government has certain rights in the invention.

### BACKGROUND OF THE INVENTION

Heart disease remains the leading cause of death in the United States, with 1 in 4 deaths being a attributable to the disease [1,2]. Among one of the most common heart diseases is Hypertrophic Cardiomyopathy (HCM), which affects 1 in 500 people and is a leading cause of spontaneous cardiac death in young adults [3]. Patients with HCM exhibit a diverse set of symptoms, though a hypertrophied myocardium, hypercontractility, and reduced ejection volume are among the most common [4]-[6]. Additionally, in HCM the myocardium undergoes remodeling as cardiac fibroblasts deposit excessive extracellular matrix (ECM), leading to fibrosis (increased stiffness of the heart) [7].

A current barrier in understanding the mechanisms of HCM has been the lack of an appropriate model that effectively mimics the native structure and mechanobiology of human adult cardiomyocytes (CMs). CMs are the cells responsible for heart contraction. The ideal CM model would be cardiomyocytes directly isolated from a patient, however primary adult CMs do not regenerate and are difficult to culture in vitro [8]. Other models include primary CMs derived from animals such as rats, mice, chicken, and zebrafish[13-16]. An advantage to these is that they can be used as genetic. models to obtain information such as protein function and mechanisms behind the progression of cardiovascular diseases [13]. However, these models do not accurately recapitulate mechanisms of maladaptive remodeling in humans [14]. Further, they have different responses to pharmaceutics, are not suitable for long-term studies, and their physiology differs significantly from human physiology [17, 19]. Human induced pluripotent stem cell derived cardiomyocytes (hiPSC-CMs) have gained significant traction over the last decade as a powerful model for understanding cardiac development, modeling cardiac diseases, drug screening, and cardiotoxicity screening[16]. These stem cells are derived from patient somatic cells, reprogrammed to a pluripotent state, and then differentiated into cardiomyocytes [17]. They hold great promise for personalized medicine and can be genetically edited to display various mutations linked to diseases, making them an attractive model [18].

Despite the potential of hiPSC-CMs as a powerful model, they are limited by the immaturity and heterogeneity that is observed not only across different lab groups and lab members, but also across batch to batch even when using the same protocol [23, 24]. hiPSC-CMs display a fetal-like phenotype in terms of sarcomere structure, t-tubule organization, metabolism, calcium handling, and overall morphology [21]. Current methods to improve CM maturity are prolonged culture time, addition of biochemical cues, biophysical stimulation, altering substrate stiffness and/or extracellular matrix proteins [22]. According to previous studies, the in vitro microenvironment can have a drastic effect on hiPSC-CM maturation and promote a more adult, rod-like CM structure and organized sarcomeres [22].

Cells are known to sense their local microenvironment properties via mechanosensitive adhesion proteins and their associated protein complexes [23]. These adhesion complexes serve as bidirectional mechanical linkages that can transmit intracellular signals that result in changes in gene transcription, morphology, and function. Studies have shown that native CM cytoskeleton structure, anisotropic contraction direction, and contractility can be recapitulated by manipulating the microenvironment. Thus, there is a need to provide a more physiologically relevant microenvironment in in vitro studies. Parameters that can be altered in vitro include ECM protein type, substrate stiffness, and linker type.

Amongst the most common methods currently used to culture hiPSC-CMs in vitro is a monolayer of CMs cultured on polystyrene tissue culture plastic that has been physiosorbed with ECM proteins such as Laminin, Fibronectin, Collagen, or Matrigel [19], [24]-[26]. However, tissue culture plastic lacks key features of the native myocardium, for instance it has a stiffness of 3 GPa, which is about six orders of magnitude stiffer than that of the native myocardium [30,23]. Further, some studies have even reported structural damage and loss of contractility [28]. A more physiological relevant alternative is using hydrogels, which are linearly elastic, transparent, highly tunable and can be optimized for different purposes. Hydrogels can be functionalized with various ECM protein types and their stiffness can be tuned, both of which have been shown to modulate CM attachment, spread area, differentiation lineage, and contractility [29-32]. Further, fiducial microbeads can be embedded into hydrogels to allow for functional contractility experiments such as traction force microscopy (TFM) [33].

As previously mentioned, hiPSC-CM's display an immature phenotype in terms of morphology [21]. Whereas adult cardiomyocytes display a rod-shaped morphology, immature hiPSC-CMs display a more circular morphology [22].

The cell's native morphology can be achieved in vitro with surface patterning techniques. Patterning results in a substrate with discrete ECM “islands” to which cells can adhere to, and these regions are surrounded with proteins or polymers to which cells are unable to adhere to. An in vitro study seeded single cell hiPSC-CMs on rectangular micropatterns with various aspect ratios and observed significant differences in myofibril organization and contraction force magnitude [33]. hiPSC-CMs on 7:1 aspect ratio patterns showed an increase in myofibril alignment, sarcomere activity, and mechanical output compared to smaller aspect ratios (3:1 and 1:1), or unpatterned.

There remains a need for better methods of producing patterned substrates for use with cells.

### SUMMARY OF THE INVENTION

A method for performing protein patterning is provided. In particular, the method comprises generating a photoresist pattern on a wafer using photolithography; dicing the photoresist patterned wafer into wafer chips; and performing lift-off protein patterning on the photoresist patterned wafer chips. This manufacturing technique scales up the photolithography stage of the lift-off fabrication workflow to generate high quantities of shelf-stable photoresist patterned glass substrates, which can be used to incorporate a protein pattern onto the surface of a hydrogel. Key novel features of the manufacturing method include (1) wafer scale fabrication and optimization for dicing saw processing, (2) a shelf-stable intermediate stage, (3) optimization of the process for transfer of protein patterning to a hydrogel via the addition of spacers and (4) demonstration of compatibility with cells, including human induced pluripotent stem cell cardiomyocytes.

In one aspect, a method for performing protein patterning is provided, the method comprising: a) generating a photoresist pattern on a wafer using photolithography; b) dicing the photoresist patterned wafer to obtain a plurality of photoresist patterned wafer chips; and c) performing lift-off protein patterning on the plurality of photoresist patterned wafer chips to produce a protein pattern on the plurality of wafer chips.

In certain embodiments, the method further comprises transferring the protein pattern from the plurality of wafer chips to a hydrogel. In some embodiments, transferring the protein pattern from the plurality of wafer chips to a hydrogel comprises: placing a protein patterned wafer chip on top of a spacer with the protein pattern facing downwards toward the spacer, wherein the spacer controls thickness of the hydrogel that forms; adding a solution comprising a hydrogel polymer between the protein patterned wafer chip and the spacer; polymerizing the hydrogel polymer to form the hydrogel, wherein the protein pattern is transferred from the protein patterned wafer chip to the hydrogel; and removing the wafer chip from the hydrogel.

In certain embodiments, the method further comprises adding a cell to the hydrogel, wherein the cell adheres to a protein in the protein pattern.

In certain embodiments, the compressive modulus of the hydrogel ranges from 1 kPa to 35 kPa, including any value within this range such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 35 kPa. In some embodiments, the hydrogel has a stiffness ranging from about 5 kPa to 10 kPa.

In certain embodiments, the hydrogel comprises polyacrylamide.

In certain embodiments, the method of lift-off protein patterning comprises: incubating the plurality of photoresist patterned wafer chips with poly(l-lysine)-graft-poly (ethylene glycol) (PLL-g-PEG) copolymer; removing the photoresist from the plurality of wafer chips; and incubating the plurality of wafer chips with a protein of interest, wherein the protein of interest forms a pattern on the plurality of wafer chips. The photoresist can be removed from a wafer chip, for example, by submerging the wafer chips in an aqueous solution comprising N-methyl pyrrolidone (see Example 1).

In certain embodiments, the wafer has a thickness ranging from about 250 μm to about 700 μm, including any thickness within this range such as 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, or 700 μm. In some embodiments, the wafer has a thickness of about 500 μm.

In certain embodiments, the wafer has a diameter ranging from about 50 mm to about 300 mm, including any diameter within this range such as 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, 290 mm, or 300 mm. In some embodiments, the wafer has a diameter of about 100 mm.

In certain embodiments, the wafer is a glass wafer or a silicon wafer. In some embodiments, the wafer further comprises an oxide layer.

In certain embodiments, the method further comprises protecting the photoresist pattern from light exposure during said dicing by covering the wafer with an opaque material (e.g., opaque tape such as masking tape).

In certain embodiments, the wafer is diced with a dicing saw (e.g., with a thermocarbon diamond blade).

In certain embodiments, the wafer is diced into at least 16 chips per wafer.

In certain embodiments, the wafer chips are square or rectangular. For example, the wafer can be diced into square chips with a side having a length of about 15 mm.

In certain embodiments, lift-off protein patterning is performed with one or more extracellular matrix proteins. Exemplary extracellular matrix proteins include, without limitation, laminin, fibronectin, collagen, elastin, nidogen, heparan sulfate, chondroitin sulfate, keratan sulfate, or hyaluronic acid.

In certain embodiments, the method further comprises storing the photoresist patterned wafer or the photoresist patterned wafer chips for a period of time prior to said performing lift-off protein patterning.

### BRIEF DESCRIPTION OF THE DRAWINGS

FIG. 1. Wafer fabrication process of 4″ glass wafers using photolithography, from photoresist deposition to dicing to obtain individual glass chips, and development of the photoresist. Developed glass chips can be stored for at least six months in a light-protected environment prior to the fabrication of hydrogel devices.

FIG. 2. Protocol for generating protein patterns on glass chips by lift-off and fabrication of hydrogel devices with protein patterns by transfer method.

FIGS. 3A-3B. Transfer of protein templates in photoresist on glass chips into protein patterns on hydrogels. FIG. 3A) Developed photoresist patterns on glass chips. FIG. 3B) Protein patterns on hydrogel devices were visualized using fluorescent gelatin. Fluorescent gelatin was transferred from glass chips using PDMS spacers to define the hydrogel thickness. Scale bars, 25 μm.

### DETAILED DESCRIPTION

A method for performing protein patterning is provided. In particular, the method comprises generating a photoresist pattern on a wafer using photolithography; dicing the photoresist patterned wafer into wafer chips; and performing lift-off protein patterning on the photoresist patterned wafer chips. This manufacturing technique scales up the photolithography stage of the lift-off fabrication workflow to generate high quantities of shelf-stable photoresist patterned glass substrates, which can be used to incorporate a protein pattern onto the surface of a hydrogel.

Before the present protein patterning methods are described, it is to be understood that this invention is not limited to the particular methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the chip” includes reference to one or more chips and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The term “hydrogel” refers to a substance formed when an organic polymer (natural or synthetic) is cross-linked via covalent, ionic, or hydrogen bonds to create a three-dimensional open-lattice structure which entraps water molecules to form a gel. In general, these polymers are at least partially soluble in aqueous solutions, such as water, buffered salt solutions, or aqueous alcohol solutions that have charged side groups, or a monovalent ionic salt thereof. Biocompatible hydrogel refers to a polymer that forms a gel which is not toxic to living cells, and allows sufficient diffusion of oxygen and nutrients to the entrapped cells to maintain viability.

The phrase “mammalian cell” refers to any cell originating from mammalian tissue. The cell can be a primary cell obtained directly from a mammalian subject. The cell may also be a cell derived from the culture and expansion of a cell obtained from a subject. For example, the cell may be a stem cell, progenitor cell, or adult cell. Immortalized cells are also included within this definition. In some embodiments, the cell has been genetically engineered to express a recombinant protein and/or nucleic acid. The term “mammalian” includes, without limitation, human, equine, bovine, porcine, canine, feline, rodent (e.g., mice, rats, hamster), and primate.

“Biocompatible,” as used herein, refers to a property of a material that allows for prolonged contact with a cell or tissue without causing toxicity or significant damage.

“Substantially purified” generally refers to isolation of a substance (compound, polynucleotide, protein, polypeptide, peptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

### Protein Patterning

In one aspect, a method for performing protein patterning is provided. The method comprises: a) generating a photoresist pattern on a wafer using photolithography; b) dicing the photoresist patterned wafer to obtain a plurality of photoresist patterned wafer chips; and c) performing lift-off protein patterning on the plurality of photoresist patterned wafer chips to produce a protein pattern on the plurality of wafer chips. In certain embodiments, the method further comprises transferring the protein pattern from the plurality of wafer chips to a hydrogel.

An advantage of this method is that multiple patterned chips can be generated uniformly, which allows consistent protein patterning on hydrogels. This method facilitates production of multiple patterned hydrogels with different properties in terms of stiffness or choice of proteins used in patterning. The glass chips are compatible with different types of cell culture substrates, including glass coverslips and multi-well plates. Additionally, the photoresist patterned wafers or photoresist patterned wafer chips can be stored for a period of time prior to performing lift-off protein patterning, which adds to the convenience of manufacturing protein patterned hydrogels.

The wafer used in the practice of the subject methods may have a thickness ranging from about 250 μm to about 700 μm, including any thickness within this range such as 250 μm, 275 μm, 300 μm, 325 μm, 350 μm, 375 μm, 400 μm, 425 μm, 450 μm, 475 μm, 500 μm, 525 μm, 550 μm, 575 μm, 600 μm, 625 μm, 650 μm, 675 μm, or 700 μm. In some embodiments, the wafer has a thickness of about 500 μm. In certain embodiments, the wafer has a diameter ranging from about 50 mm to about 300 mm, including any diameter within this range such as 50 mm, 60 mm, 70 mm, 80 mm, 90 mm, 100 mm, 110 mm, 120 mm, 130 mm, 140 mm, 150 mm, 160 mm, 170 mm, 180 mm, 190 mm, 200 mm, 210 mm, 220 mm, 230 mm, 240 mm, 250 mm, 260 mm, 270 mm, 280 mm, 290 mm, or 300 mm. In some embodiments, the wafer has a diameter of about 100 mm. In certain embodiments, the wafer is a glass wafer or a silicon wafer. In some embodiments, the wafer further comprises an oxide layer. Suitable wafers that can be used in the subject methods are commercially available, for example, from University Wafer (South Boston, Mass.).

The photoresist pattern can be created on the wafer using standard photolithography techniques known in the art. For example, photoresist can be applied to the wafer by spin-coating, followed by exposure of the photoresist to a pattern of light, post-exposure baking, and development of the photoresist. Plasma treatment may be omitted to avoid changes to the surface properties of the material and detachment of the photoresist at the development step. In some embodiments, wafers are cleaned prior to applying the photoresist, for example, with acetone, isopropanol, and water. The wafers can be dried using nitrogen gas and then dehydrated on a hotplate. See, e.g., Example 1 for a description of a method of spin-coating a positive photoresist (e.g., AZ1512) onto a wafer. The photoresist can be spun first at 500 rpm for 10 seconds, then ramped up to 2000 rpm for 45 seconds, followed by exposure of the photoresist using a Karl Suss MA6 aligner at 50 mJ/cm² with a bright-field mask on transparency (CAD/Art Services, Inc). Soft or hard contact modes can be used to extend the lifetime of the mask. For a description of photolithography techniques, see, e.g., Mack Fundamental Principles of Optical Lithography: The Science of Microfabrication, Wiley, 2007; and Levinson Principles of Lithography, Society of Photo Optical, 4^{th} edition, 2019; herein incorporated by reference.

The photoresist pattern on the wafer can be protected from light exposure after performing photolithography by covering the wafer with an opaque material. For example, the photoresist pattern on the wafer can be covered with an opaque tape such as masking tape prior to dicing.

The wafer can be diced with a dicing saw, e.g., having a thermocarbon diamond blade. In certain embodiments, the wafer is diced into at least 10 chips, at least 12 chips, at least 14 chips, at least 15 chips, at least 16 chips, at least 17 chips, at least 18 chips, at least 19, or at least 20 chips per wafer. In certain embodiments, the wafer chips are square or rectangular. For example, the wafer can be diced into square chips with a side having a length of about 10 mm to about 20 mm, including any length within this range such as 10 mm, 11 mm, 12 mm, 13 mm, 14 mm, 15 mm, 16 mm, 17 mm, 18 mm, 19 mm, or 20 mm. In some embodiments, the wafer is diced into square chips with a side length of about 15 mm.

Protein patterning may be performed with one or more proteins. In certain embodiments, lift-off protein patterning is performed with one or more extracellular matrix proteins. Exemplary extracellular matrix (ECM) proteins include, without limitation, laminin, fibronectin, collagen, elastin, nidogen, heparan sulfate, chondroitin sulfate, keratan sulfate, or hyaluronic acid. In some embodiments, protein patterning is performed with a composition comprising basement matrix proteins such as Matrigel. In some embodiments, protein patterning of ECM proteins is designed to mimic the spatial organization of the extracellular matrix of the cell microenvironment in vivo.

The photoresist pattern is used to produce a protein pattern on the wafer chips using lift-off protein patterning. In certain embodiments, the method of lift-off protein patterning comprises: incubating the plurality of photoresist patterned wafer chips with poly(l-lysine)-graft-poly (ethylene glycol) (PLL-g-PEG) copolymer; removing the photoresist from the plurality of wafer chips; and incubating the plurality of wafer chips with a protein of interest, wherein the protein of interest forms a pattern on the plurality of wafer chips. The photoresist can be removed from a wafer chip, for example, by submerging the wafer chips in an aqueous solution comprising N-methyl-2-pyrrolidone (see e.g., Example 1 and Moeller et al. (2018) PLoS One 13(1):e0189901, herein incorporated by reference in its entirety).

### Hydrogels

The protein pattern on a wafer chip can be transferred to a hydrogel. In some embodiments, the method of transfer of the protein pattern from a wafer chip to the hydrogel comprises placing a protein patterned wafer chip on top of a spacer with the protein pattern facing downwards toward the spacer; adding a solution comprising a hydrogel polymer between the protein patterned wafer chip and the spacer; and polymerizing the hydrogel polymer to form the hydrogel, wherein the protein pattern is transferred from the protein patterned wafer chip to the hydrogel. The spacer is used to control the thickness of the hydrogel that forms. The wafer chip can be removed after the hydrogel polymerizes.

Any suitable hydrogel polymers can be used to form a hydrogel. Exemplary hydrogel polymers include, without limitation, natural polymers such as polysaccharides, including hyaluronic acid, chitosan, heparin, alginate, cellulose, dextran, and agarose, and proteins, including fibrin, fibrinogen, collagen, elastin, gelatin, silk, laminin, fibronectin, albumin, thrombin, and keratin; modified natural polymers, including hydroxymethylcellulose, hydroxyethylcellulose, gelatin methacrylate, polyanionic N-carboxymethyl chitosan, and polycationic N-trimethyl chitosan; and synthetic polymers, including polyvinyl alcohol, N-vinylpyrrolidone, polyethylene glycol, poly(ethylene glycol) diacrylate, polyacrylamide, poly(N-isopropylacrylamide), sodium polyacrylate, acrylate polymers and copolymers such as hydroxyethyl methacrylate, ethyl methacrylate, propylene glycol methacrylate, ethylene glycol di-methyl acrylate, methyl methacrylate, glycidyl methacrylate, and glycol methacrylate, poly(N-isopropylacrylamide-co-acrylic acid), polyesters, polyurethanes, nylon, synthetic polyamino acids, prolamines; and combinations thereof, and other such molecules, including recombinant versions of such polymers.

Methods of preparing hydrogels are well known in the art. See, e.g., Barbucci Hydrogels Biological Properties and Applications, Springer, 2009; Hydrogels in Cell-Based Therapies, edited by Connon and Hamley, Royal Society of Chemistry, 2014; Tunable Hydrogels, edited by Lavrentieva, Pepelanov, and Seliktar, Springer Nature Switzerland AG, 2020; herein incorporated by reference. In certain instances, a hydrogel is crosslinked by chemical crosslinking. Exemplary chemical crosslinking agents include, without limitation, glutaraldehyde, formaldehyde, epoxy compounds, dialdehyde, N,N′-methylenebis(acrylamide) (MBA), ethylene glycol diacrylate (EGDA), ethylene glycol dimethylacrylate (EGDMA), PEG diacrylate (PEGDA), glyoxal, epichlorohydrin, and sodium borate/boric acid. The crosslink density may vary depending on the type and the concentration of the chemical crosslinking agent employed. Alternatively, the hydrogel may be photo-crosslinked by exposure to light. UV light-sensitive photoinitiators (i.e., initiate polymerization with exposure to UV light in the range of 200-400 nm) or visible light-sensitive photoinitiators (i.e., initiate polymerization with exposure to visible light in the range of 400-800 nm) may be used. A photoinitiator may be used to induce photopolymerization. Photopolymerization reactions may involve a free-radical-initiated chain polymerization or bio-orthogonal click reactions. Exemplary photoinitiators include, without limitation, (1-[4-(2-hydroxyethoxy)-phenyl]-2-hydroxy-2-methyl-1-propane-1-one), lithium phenyl-2,4,6-trimethylbenzoylphosphinate (LAP), and Eosin-Y. The crosslink density may vary depending on the intensity of the electromagnetic radiation applied to promote photopolymerization of the hydrogel as well as the duration of irradiation. In some embodiments, the crosslink density of the crosslinked hydrogel ranges from 1×10⁻¹⁵ moles/cm³ to 1×10⁻³ moles/cm³. Accordingly, depending on the amount of crosslinking, the swelling ratio of the subject hydrogels may vary, ranging, for example, from 1 to 35.

The compressive modulus (i.e., material stiffness, also referred to herein as “modulus”) of a hydrogel can be in the range of from 5 pascals (Pa) to 500 kilopascals (kPa) (e.g., from 100 Pa to 400 kPa, from 300 Pa to 400 kPa, from 300 Pa to 50 kPa, from 300 Pa to 30 kPa, from 300 Pa to 20 kPa, from 300 Pa to 15 kPa, from 400 Pa to 300 kPa, from 500 Pa to 200 kPa, from 500 Pa to 100 kPa, from 500 Pa to 50 kPa, from 500 Pa to 25 kPa, from 500 Pa to 20 kPa, from 500 Pa to 15 kPa, from 500 Pa to 10 kPa, from 750 Pa to 10 kPa, from 800 Pa to 10 kPa, from 900 Pa to 10 kPa, from 1 kPa to 10 kPa, from 2 kPa to 10 kPa, from 3 kPa to 10 kPa, from 2 kPa to 9 kPa, from 3 kPa to 9 kPa, from 500 Pa to 5 kPa, from 500 Pa to 3 kPa, or from 500 Pa to 2 kPa). In some embodiments, the compressive modulus ranges from 0.1 kPa to 40 kPa, including any value within this range such as 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 kPa. In some embodiments, the hydrogel has a stiffness ranging from about 5 kPa to 10 kPa. In some embodiments, a hydrogel is selected with a compressive modulus designed to replicate the stiffness of a particular tissue.

In certain embodiments, one or more additional factors, such as nutrients, cytokines, growth factors, antibiotics, antioxidants, or immunosuppressive agents may be added to the hydrogel to improve cell function or viability. The composition may also further comprise a pharmaceutically acceptable carrier.

Exemplary growth factors include, without limitation, fibroblast growth factor (FGF), insulin-like growth factor (IGF), transforming growth factor beta (TGF-β), epiregulin, epidermal growth factor (EGF), endothelial cell growth factor (ECGF), nerve growth factor (NGF), leukemia inhibitory factor (LIF), bone morphogenetic protein-4 (BMP-4), hepatocyte growth factor (HGF), vascular endothelial growth factor (“VEGF”), and cholecystokinin octapeptide.

Exemplary immunosuppressive agents are well known and may be steroidal (e.g., prednisone) or non-steroidal (e.g., sirolimus (Rapamune, Wyeth-Ayerst Canada), tacrolimus (Prograf, Fujisawa Canada), and anti-IL2R daclizumab (Zenapax, Roche Canada). Other immunosuppressant agents include 15-deoxyspergualin, cyclosporin, methotrexate, rapamycin, Rapamune (sirolimus/rapamycin), FK506, or Lisofylline (LSF).

In some embodiments, cells are grown (i.e., cultured) on a subject hydrogel matrix for any convenient amount of time (e.g., 5 minutes to 2 weeks, e.g., 5-30 minutes, 5 minutes, 10 minutes, 15 minutes, 20 minutes, 30 minutes, 30 minutes to 1 hour, 1 hour, 1-2 hours, 2 hours, 1-3 hours, 3 hours, 2-4 hours, 4 hours, 3-5 hours, 5 hours, 4-6 hours, 6 hours, 8 hours, 10 hours, 12-24 hours, 12 hours, 18 hours, 24 hours, 24-36 hours, 36 hours, 36-48, 48 hours, 2 days-1 week, 2 days, 3 days, 4 days, 5 days, 6 days, 1 week, 1-2 weeks, or 2 weeks) under any desired condition.

### Cultures

In some embodiments, cells or tissue are obtained from a subject for the purpose of growing cultures of cells, populations of cells, or tissue in a protein patterned hydrogel, prepared as described herein. The cells may be derived from any tissue, including connective tissue, muscle tissue, nervous tissue, or epithelial tissue. Cells or tissue may be obtained by any convenient method including, without limitation, by biopsy, e.g., during endoscopy, during surgery, by needle, etc., and are preferably obtained as aseptically as possible. In some embodiments, the cells or tissue are from a mammalian species such as, but not limited to a human, equine, bovine, porcine, canine, feline, rodent (e.g., mice, rats, hamster), or primate subject. The subject may be of any age, e.g., a fetus, neonate, juvenile, or adult.

Cells used in cultures can be primary cells obtained directly from a subject. Alternatively, the cells may be derived from the culture and expansion of a cell obtained from a subject or a cell obtained from a cell line. In some embodiments, the cell is an adult cell. In other embodiments, the cell is a progenitor cell or stem cell, or a differentiated cell derived from a progenitor cell or stem cell. Immortalized cells may also be used in cultures. In some embodiments, the cell has been genetically engineered to express a recombinant protein and/or nucleic acid.

Cells or tissue used in cultures may be obtained from any part of the body of a subject, including, without limitation, from the cardiovascular system, including the heart, blood, blood vessels, and lungs; digestive system, including the salivary glands, esophagus, stomach, liver, gallbladder, pancreas, intestines, colon, rectum and anus; endocrine system, including the endocrine glands such as the hypothalamus, pituitary gland, pineal body or pineal gland, thyroid, parathyroids and adrenals (adrenal glands); excretory system, including kidneys, ureters, bladder and urethra involved in fluid balance, electrolyte balance and excretion of urine; lymphatic system, including structures involved in the transfer of lymph between tissues and the blood stream, the lymph and the nodes and vessels that transport it, the immune system, including leukocytes, tonsils, adenoids, thymus and spleen; integumentary system, including skin, hair and nails of mammals, and scales of fish, reptiles, and birds, and feathers of birds; muscular system, including skeletal, smooth and cardiac muscles; nervous system, including the brain, spinal cord, nerves, and glia; reproductive system, including the sex organs, such as ovaries, fallopian tubes, uterus, vulva, vagina, testes, vas deferens, seminal vesicles, prostate and penis; respiratory system, including the organs used for breathing, the pharynx, larynx, trachea, bronchi, lungs and diaphragm; skeletal system, including bones, cartilage, ligaments and tendons.

Cells included in cultures may be of any type such as, but not limited to, exocrine secretory epithelial cells such as a Brunner's gland cell in the duodenum, insulated goblet cell of respiratory and digestive tracts, stomach cells such as foveolar cell (mucus secretion), a chief cell (pepsinogen secretion), parietal cell (hydrochloric acid secretion), and pancreatic acinar cell; a Paneth cell of the small intestine, a type II pneumocyte of lung, a club cell of the lung; barrier cells such as a type I pneumocyte (lung), gall bladder epithelial cell, centroacinar cell (pancreas), intercalated duct cell (pancreas), and intestinal brush border cell (with microvilli); hormone-secreting cells such as an enteroendocrine cell, K cell, L cell, I cell, G cell, enterochromaffin cell, enterochromaffin-like cell, N cell, S cell, D cell, Mo cell, thyroid gland cells, thyroid epithelial cell, parafollicular cell, parathyroid gland cells, parathyroid chief cell, oxyphil cell, pancreatic islets (islets of Langerhans), alpha cell (secretes glucagon), beta cell (secretes insulin and amylin), delta cell (secretes somatostatin), epsilon cell (secretes ghrelin), pp cell (gamma cell), cells derived primarily from ectoderm such as exocrine secretory epithelial cells, salivary gland mucous cell, salivary gland serous cell, von Ebner's gland cell in tongue, mammary gland cell, lacrimal gland cell, ceruminous gland cell in ear, eccrine sweat gland dark cell, eccrine sweat gland clear cell, apocrine sweat gland cell, gland of moll cell in eyelid, sebaceous gland cell, and bowman's gland cell in nose; hormone-secreting cells such as anterior/intermediate pituitary cells, corticotropes, gonadotropes, lactotropes, melanotropes, somatotropes, thyrotropes, magnocellular neurosecretory cells, parvocellular neurosecretory cells, and chromaffin cells (adrenal gland); epithelial cells such as a keratinocyte, epidermal basal cell, melanocyte, trichocyte, medullary hair shaft cell, cortical hair shaft cell, cuticular hair shaft cell, Huxley's layer hair root sheath cell, Henle's layer hair root sheath cell, outer root sheath hair cell, surface epithelial cell of cornea, tongue, mouth, nasal cavity, distal anal canal, distal urethra, and distal vagina, basal cell (stem cell) of cornea, tongue, mouth, nasal cavity, distal anal canal, distal urethra, and distal vagina, intercalated duct cell (salivary glands), striated duct cell (salivary glands), lactiferous duct cell (mammary glands), ameloblast, oral cells such as an odontoblast and cementoblast; nervous system cells such as neurons, sensory transducer cells such as auditory inner hair cells of organ of Corti, auditory outer hair cells of organ of Corti, basal cells of olfactory epithelium, cold-sensitive primary sensory neurons, heat-sensitive primary sensory neurons, merkel cells of epidermis, olfactory receptor neurons, pain-sensitive primary sensory neurons, photoreceptor cells of retina in the eye such as photoreceptor rod cells, photoreceptor blue-sensitive cone cells of eye, photoreceptor green-sensitive cone cells of eye, and photoreceptor red-sensitive cone cells of eye; proprioceptive primary sensory neurons, touch-sensitive primary sensory neurons, chemoreceptor glomus cells of carotid body cell, outer hair cells of vestibular system of ear, inner hair cells of vestibular system of ear, taste receptor cells of taste bud, autonomic neuron cells, cholinergic neurons, adrenergic neural cells, peptidergic neural cells, sense organ and peripheral neuron supporting cells, inner pillar cells of organ of Corti, outer pillar cells of organ of Corti, inner phalangeal cells of organ of Corti, outer phalangeal cells of organ of Corti, border cells of organ of Corti, Hensen's cells of organ of Corti, vestibular apparatus supporting cells, taste bud supporting cells, olfactory epithelium supporting cells, olfactory ensheathing cells, schwann cells, satellite glial cells, enteric glial cells, central nervous system neurons and glial cells, interneurons basket cells, cartwheel cells, stellate cells, golgi cells, granule cells, lugaro cells, unipolar brush cells, martinotti cells chandelier cells, Cajal-Retzius cells, double-bouquet cells, neurogliaform cells, retina horizontal cells, amacrine cells, starburst amacrine cells, spinal interneurons, renshaw cells, principal cells, spindle neurons, fork neurons, pyramidal cells, place cells, grid cells, speed cells, head direction cells, betz cells, stellate cells, boundary cells, bushy cells, Purkinje cells, medium spiny neurons, astrocytes, oligodendrocytes, ependymal cells, tanycytes, pituicytes, lens cells, anterior lens epithelial cell, crystallin-containing lens fiber cell; metabolism and storage cells such as adipocytes, white fat cell, brown fat cell, and liver lipocyte; secretory cells such as cells of the adrenal cortex, cells of the zona glomerulosa produce mineralocorticoids, cells of the zona fasciculata produce glucocorticoids, cells of the zona reticularis produce androgens, theca interna cell of ovarian follicle secreting estrogen, corpus luteum cell of ruptured ovarian follicle secreting progesterone, granulosa lutein cells, theca lutein cells, leydig cell of testes secreting testosterone, seminal vesicle cell, prostate gland cell, bulbourethral gland cell, Bartholin's gland cell, gland of littre cell, uterus endometrium cell, juxtaglomerular cell, macula densa cell of kidney, peripolar cell of kidney, and mesangial cell of kidney; urinary system cells such as parietal epithelial cell, podocyte, proximal tubule brush border cell, loop of henle thin segment cell, kidney distal tubule cell, kidney collecting duct cell, principal cell, intercalated cell, and transitional epithelium (lining urinary bladder); reproductive system cells such as duct cell (of seminal vesicle, prostate gland, etc.), efferent ducts cell epididymal principal cell, and epididymal basal cell; circulatory system cells, endothelial cells, extracellular matrix cells, planum semilunatum epithelial cell of vestibular system of ear, organ of Corti interdental epithelial cell, loose connective tissue fibroblasts, corneal fibroblasts (corneal keratocytes) tendon fibroblasts, bone marrow reticular tissue fibroblasts, other nonepithelial fibroblasts, pericyte, hepatic stellate cell (ito cell), nucleus pulposus cell of intervertebral disc, hyaline cartilage chondrocyte, fibrocartilage chondrocyte, elastic cartilage chondrocyte, osteoblast/osteocyte, osteoprogenitor cell, hyalocyte of vitreous body of eye, stellate cell of perilymphatic space of ear, and pancreatic stellate cell; contractile cells such as skeletal muscle cells, red skeletal muscle cell (slow twitch), white skeletal muscle cell (fast twitch), intermediate skeletal muscle cell, nuclear bag cell of muscle spindle, nuclear chain cell of muscle spindle, myosatellite cell (stem cell), cardiac muscle cells, cardiac muscle cell, SA node cell, Purkinje fiber cell, smooth muscle cell (various types) myoepithelial cell of iris myoepithelial cell of exocrine glands; blood and immune system cells such as an erythrocyte (red blood cell) and precursor erythroblasts megakaryocyte (platelet precursor) platelets, a monocyte, connective tissue macrophage (various types), epidermal langerhans cell osteoclast (in bone), dendritic cell (in lymphoid tissues), microglial cell (in central nervous system), neutrophil granulocyte and precursors (myeloblast, promyelocyte, myelocyte, metamyelocyte), an eosinophil granulocyte and precursors basophil granulocyte and precursors, a mast cell, helper T cell, regulatory T cell, cytotoxic T cell, natural killer T cell, B cell, plasma cell, natural killer cell, and hematopoietic stem cells; germ cells such as an oogonium/oocyte, spermatid, spermatocyte, spermatogonium cell, spermatozoon, nurse cell, granulosa cell, sertoli cell, and epithelial reticular cell; and interstitial cells such as interstitial kidney cells.

In some embodiments, the cells are stem cells or stem cell-derived cells. Stem cells of interest include, without limitation, hematopoietic stem cells, embryonic stem cells, mesenchymal stem cells, neural stem cells, epidermal stem cells, endothelial stem cells, gastrointestinal stem cells, liver stem cells, cord blood stem cells, amniotic fluid stem cells, skeletal muscle stem cells, smooth muscle stem cells (e.g., cardiac smooth muscle stem cells), pancreatic stem cells, olfactory stem cells, hematopoietic stem cells, induced pluripotent stem cells; and the like; as well as differentiated cells that can be cultured in vitro and used in a therapeutic regimen, where such cells include, but are not limited to, keratinocytes, adipocytes, cardiomyocytes, neurons, osteoblasts, pancreatic islet cells, retinal cells, and the like.

Suitable human embryonic stem (ES) cells include, but are not limited to, any of a variety of available human ES lines, e.g., BG01 (hESBGN-01), BG02 (hESBGN-02), BG03 (hESBGN-03) (BresaGen, Inc.; Athens, Ga.); SA01 (Sahlgrenska 1), SA02 (Sahlgrenska 2) (Cellartis AB; Goeteborg, Sweden); ES01 (HES-1), ES01 (HES-2), ES03 (HES-3), ES04 (HES-4), ES05 (HES-5), ES06 (HES-6) (ES Cell International; Singapore); UC01 (HSF-1), UC06 (HSF-6) (University of California, San Francisco; San Francisco, Calif.); WA01 (H1), WA07 (H7), WA09 (H9), WA09/Oct4D10 (H9-hOct4-pGZ), WA13 (H13), WA14 (H14) (Wisconsin Alumni Research Foundation; WARF; Madison, Wis.). Cell line designations are given as the National Institutes of Health (NIII) code, followed in parentheses by the provider code.

Hematopoietic stem cells (HSCs) are mesoderm-derived cells that can be isolated from bone marrow, blood, cord blood, fetal liver and yolk sac. HSCs are characterized as CD34⁺ and CDT. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

Mesenchymal stem cells (MSC), originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC.

An induced pluripotent stem (iPS) cells is a pluripotent stem cell induced from a somatic cell, e.g., a differentiated somatic cell. iPS cells are capable of self-renewal and differentiation into cell fate-committed stem cells, including neural stem cells, as well as various types of mature cells. iPS cells can be generated from somatic cells, including skin fibroblasts, using, e.g., known methods. iPS cells can be generated from somatic cells (e.g., skin fibroblasts) by genetically modifying the somatic cells with one or more expression constructs encoding Oct-3/4 and Sox2. In some embodiments, somatic cells are genetically modified with one or more expression constructs comprising nucleotide sequences encoding Oct-3/4, Sox2, c-myc, and K1f4. In some embodiments, somatic cells are genetically modified with one or more expression constructs comprising nucleotide sequences encoding Oct-4, Sox2, Nanog, and LIN28. Methods of generating iPS are known in the art, and any such method can be used to generate iPS.

In some cases, the cells are lymphocytes, such as CD4+ and/or CD8+ T lymphocytes, or B lymphocytes. In some embodiments, the therapeutic cells are cytotoxic T lymphocytes. In some embodiments, the lymphocytes are genetically modified lymphocytes, e.g., chimeric antigen receptor (CAR) T lymphocytes. The lymphocytes, e.g., cytotoxic T lymphocytes, may specifically recognize an antigen that is associated with a disease, e.g., cancer or tumor.

In some embodiments, the cells include insulin-secreting cells. The insulin-secreting cells may be any suitable type of insulin-secreting cell. In some cases, the insulin-secreting cells are a type of cell that secretes insulin (e.g., pancreatic β islet cells, or β-like cells). In some cases, the insulin-secreting cells are primary β islet cells (e.g., mature β islet cells isolated from a pancreas). In some cases, the insulin-secreting cells are β cells, or β-like cells that are derived in vitro from immature cell, precursor cells, progenitor cells, or stem cells. The insulin-secreting cells may be derived from (i.e., obtained by differentiating) stem and/or progenitor cells such as hepatocytes (e.g., transdifferentiated hepatocytes), acinar cells, pancreatic duct cells, stem cells, embryonic stem cells (ES), partially differentiated stem cells, non-pluripotent stem cells, pluripotent stem cells, induced pluripotent stem cells (iPS cells), etc. Suitable insulin-secreting cells and methods of generating the same are described in, e.g., US20030082810; US20120141436; and Raikwar et al. (PLoS One. 2015 Jan. 28; 10(1):e0116582), each of which are incorporated herein by reference.

Various culture media and methods of culturing cells and tissue are known in the art. See, e.g., Methods in Enzymology Volume 58 on Cell Culture, edited by N. P. Kaplan, N. P. Colowick, W. B. Jakoby, and I. H. Pastan, Academic Press, 1s^{t} edition, 1979; Freshney and Capes-Davis, Freshney's Culture of Animal Cells: A Manual of Basic Technique and Specialized Applications, Wiley-Blackwell, 8^{th} edition, 2021; 3D Cell Culture: Methods and Protocols (Methods in Molecular Biology, 695), edited by J. Haycock, Humana, 2011^{th} edition, 2010; Organoids and Mini-Organs, edited by J. Davies and M. Lawrence, Academic Press, 1^{st} edition, 2018; Organoids: Stem Cells, Structure, and Function (Methods in Molecular Biology, 1576), Springer, 1st edition, 2019; and Human Pluripotent Stem Cell Derived Organoid Models (Volume 159) (Methods in Cell Biology, Volume 159), Academic Press, 1^{st} edition, 2020; herein incorporated by reference in their entireties.

The growth of cultures in a hydrogel may be confirmed by any convenient method, e.g., phase contrast microscopy, stereomicroscopy, histology, immunohistochemistry, electron microscopy, fluorescence microscopy, etc. In some instances, cellular ultrastructure and multi-lineage differentiation may be assessed. Ultrastructure of a culture can be determined by performing hematoxylin-eosin staining, proliferating cell nuclear antigen (PCNA) staining, electron microscopy, and the like using methods known in the art. Multi-lineage differentiation can be determined by performing labeling with antibodies to terminal differentiation markers. Antibodies to detect differentiation markers are commercially available from a number of sources.

In some embodiments, the cells in cultures may be experimentally modified. For example, cells may be modified by exposure to viral or bacterial pathogens, e.g., to develop a reagent for experiments to assess the anti-viral or anti-bacterial effects of therapeutic agents. The cells may be modified by altering patterns of gene expression, e.g., by providing reprogramming factors to induce pluripotency or otherwise alter differentiation potential, or to determine the effects of a gain or loss of gene function.

### Kits

Also provided are kits comprising one or more reagents and/or tools useful in practicing the methods of the present disclosure. In certain aspects, the kits include patterning proteins (e.g., Matrigel or extracellular matrix proteins such as laminin, fibronectin, collagen, elastin, nidogen, heparan sulfate, chondroitin sulfate, keratan sulfate, or hyaluronic acid) capable of being patterned on a surface of a wafer chip and/or hydrogel, a dicing saw for dicing wafers, reagents for photolithography (e.g., photoresist), reagents for lift-off protein patterning (e.g., poly(l-lysine)-graft-poly (ethylene glycol) (PLL-g-PEG) copolymer), and spacers, hydrogel polymers, and a free radical initiator or a photoinitiator for forming a hydrogel. The kit may also include cells, culture media, incubators, microscopes, and/or other reagents or equipment for culturing cells.

In addition to the above components, the subject kits may further include (in certain embodiments) instructions for practicing the subject methods. In some embodiments, instructions for protein patterning a substrate such as wafer chips or a hydrogel are provided in the kits. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, SD drive, and the like, on which the information has been recorded. Yet another form of these instructions that may be present is a website address which may be used via the internet to access the information at a removed site.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-24 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A method for performing protein patterning, the method comprising:
a) generating a photoresist pattern on a wafer using photolithography;
b) dicing the photoresist patterned wafer to obtain a plurality of photoresist patterned wafer chips; and
c) performing lift-off protein patterning on the plurality of photoresist patterned wafer chips to produce a protein pattern on the plurality of wafer chips.
   2. The method of aspect 1, further comprising transferring the protein pattern from the plurality of wafer chips to a hydrogel.
   3. The method of aspect 2, wherein said transferring comprises:

placing a protein patterned wafer chip on top of a spacer with the protein pattern facing downwards toward the spacer, wherein the spacer controls thickness of the hydrogel that forms;

adding a solution comprising a hydrogel polymer between the protein patterned wafer chip and the spacer;

polymerizing the hydrogel polymer to form the hydrogel, wherein the protein pattern is transferred from the protein patterned wafer chip to the hydrogel; and

removing the wafer chip from the hydrogel.

4. The method of aspect 2 or 3, further comprising adding a cell to the hydrogel.
5. The method any one of aspects 2-4, wherein the hydrogel has a stiffness ranging from about 1 kPa to 35 kPa.
6. The method any one of aspects 2-5, wherein the hydrogel comprises polyacrylamide.
7. The method of any one of aspects 1-6, wherein said performing lift-off protein patterning comprises:

incubating the plurality of photoresist patterned wafer chips with poly(l-lysine)-graft-poly (ethylene glycol) (PLL-g-PEG) copolymer;

removing the photoresist from the plurality of wafer chips;

and incubating the plurality of wafer chips with a protein of interest, wherein the protein of interest forms a pattern on the plurality of wafer chips.

8. The method of aspect 7, wherein the photoresist is removed by submerging the wafer chips in an aqueous solution comprising N-methyl-2-pyrrolidone.
9. The method of any one of aspects 1-8, wherein the wafer has a thickness ranging from about 250 μm to about 700 μm.
10. The method of aspect 9, wherein the wafer has a thickness of about 500 μm.
11. The method any one of aspects 1-10, wherein the wafer has a diameter ranging from about 50 mm to about 300 mm.
12. The method of aspect 11, wherein the wafer has a diameter of about 100 mm.
13. The method any one of aspects 1-12, wherein the wafer is a glass wafer or a silicon wafer.
14. The method of aspect 13, wherein the silicon wafer further comprises an oxide layer.
15. The method any one of aspects 1-14, further comprising protecting the photoresist pattern from light exposure during said dicing by covering the wafer with an opaque material.
16. The method of aspect 15, wherein the opaque material is an opaque tape.
17. The method any one of aspects 1-16, wherein the wafer is diced with a dicing saw.
18. The method of aspect 17, wherein the dicing saw comprises a thermocarbon diamond blade.
19. The method any one of aspects 1-18, wherein said dicing produces at least 16 chips per wafer.
20. The method any one of aspects 1-19, wherein the wafer chips are square or rectangular.
21. The method of aspect 20, wherein the square has a side having a length of about 15 mm.
22. The method any one of aspects 1-21, wherein said lift-off protein patterning is performed with one or more extracellular matrix proteins.
23. The method of aspect 22, wherein the one or more extracellular matrix proteins are laminin, fibronectin, collagen, elastin, nidogen, heparan sulfate, chondroitin sulfate, keratan sulfate, or hyaluronic acid, or any combination thereof.
24. The method of any one of aspects 1-23, further comprising storing the photoresist patterned wafer or the photoresist patterned wafer chips for a period of time prior to said performing lift-off protein patterning.

### EXAMPLES

The following examples are put forth so as to provide those of ordinary skill in the art with a complete disclosure and description of how to make and use the disclosed subject matter, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1: Wafer Fabrication Process for On-demand Lift-off Protein Patterning in Multiple Hydrogels

### 1. Introduction

Here, we present a photolithography-based approach for scaled-up fabrication of glass chips that can be used to either make hydrogel devices or protein patterns directly on the chips. We focused on scaling up the photolithography process since this step in the protocol allows for a high degree of flexibility in the design and fabrication of hydrogel devices. For example, tuning mechanical properties of hydrogel devices and selection of extracellular matrix proteins occur at a later stage in the protocol. Our method can be used to generate a high quantity of pattern templates that can be used more than six months after wafer fabrication and dicing. Shelf stability of the glass chips makes it possible to externally source the pattern templates, and thus removes the requirement from labs to have cleanroom infrastructure and expertise. The remaining steps in the fabrication of hydrogel devices do not require specialized equipment besides a chemical fume hood. The scaled-up fabrication of shelf-stable chips is robust because we use 500 μm thick glass wafers, resulting in a high yield of at least 16 chips per 4″ wafer. We modified the hydrogel device fabrication method and introduced spacers to define device thickness, making it more precise and compatible with the use of glass chips. The photoresist patterns are transferred onto hydrogel devices as protein patterns for a wide range of cell culture substrates, including multi-well plates. Finally, we show that single cell hiPSC-CMs adhere, spread to a high aspect ratio, and actively contract on the hydrogels.

### 2. Materials and Methods

The final devices used for cell-seeding and imaging are protein-patterned hydrogels with a stiffness of ˜10 kPa, adhered to a cell culture substrate, in our case, a glass-bottom 6-well plate. The protein patterns inform protein interactions for the cells within a defined space; we designed the patterns to study single hiPSC-derived cardiomyocytes. The protein patterns have an area of 1500 μm² with an aspect ratio of 7:1 (length:width), which helps guide the alignment of myofibrils, thus facilitating a more mature cell phenotype. The single-cell patterns were spaced at intervals of 50 μm in x and y directions, filling the entire 5×7 inches mask so as not to require alignment during the dicing process. This section describes the fabrication process of the hydrogel devices: (1) wafer fabrication process on 4″ glass wafers using photolithography and dicing to obtain individual chips, (2) development and (3) transfer of protein patterns to hydrogel devices using lift-off and copolymerization.

### 2.1 Wafer Fabrication Process and Dicing

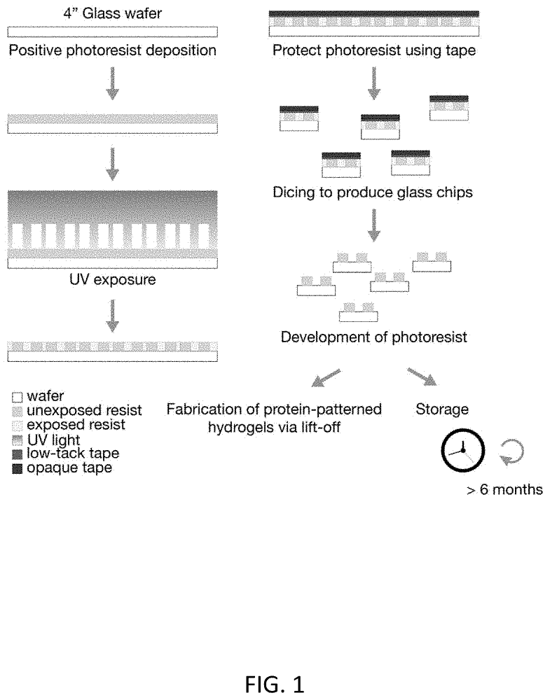

To scale up the photolithography process (summarized in FIG. 1), we selected 4″ glass wafers for their similarity in surface properties to the currently used glass microscopy coverslips (e.g., 48382-085, VWR) [34]. We chose 500 μm thick D263 glass wafers (1617, University Wafer) due to their low cost and robustness. Thinner glass wafers can also be used. We also tested silicon wafers and silicon wafers with a 1 μm oxide layer, grown in-house, for their compatibility and easy release from the dicing fixture with UV-release tape. 4″ glass wafers were thoroughly cleaned in three steps, using acetone, isopropanol, and water. Plasma treatment is not recommended as it changes surface properties of the material, and we observed this led to detachment of photoresist at the development step. The wafers were dried using a flow of nitrogen gas and then dehydrated on a hotplate for 5 minutes at 180° C. Positive photoresist AZ1512 was spun first at 500 rpm for 10 s and then ramped up to 2000 rpm for 45 s. Soft bake was done on a hotplate for 2 minutes at 100° C. The photoresist was exposed (Karl Suss MA6 aligner) at 50 mJ/cm² using a bright-field mask on transparency (CAD/Art Services, Inc). For exposure we used soft or hard contact modes to extend the lifetime of the mask.

Low-tack surface protection tape (6317A18, McMaster Carr) was gently applied on the photoresist-covered wafer, followed by clean room masking tape (76505A8, McMaster Carr) to protect the photoresist from further exposure to light. Excess tape was cut away with a microtome blade. Tape-covered wafers were diced using a dicing saw (ADT 7100, Advanced Dicing Technologies Ltd.) with a thermocarbon diamond blade (2.817-4C-30R-3), spindle speed 25 KRPM, 5 mm/s, and a reduced cut water pressure of 0.6 splm to reduce tape delamination. The 4″ glass wafer was cut 7×7 times at 0° and 90° angles. Each glass chip was 15×15 mm, yielding more than 16 chips per wafer.

### 2.2 Development

The glass wafer was attached to the dicing fixture with UV-release tape. Since the wafer is transparent with photoresist patterns, we did not use UV light to release the tape. Instead, the chips were carefully peeled away from the tape and the photoresist AZ1512 was developed in AZ 300 MIF for 60 s and rinsed with distilled water. Several chips were developed at the same time using a mini-rack holder (Z688568, Merck KGaA). Glass chips with developed photoresist can be stored in a light-protected environment for more than six months prior to lift-off protein patterning and hydrogel device fabrication.

2.3 Fabrication of Hydrogel Devices with Protein Patterns

Transfer of protein patterns to hydrogel devices using lift-off is described in detail in [34]. Briefly, glass chips with developed photoresist patterns were incubated with PLL-g-PEG (SuSoS) for 60 minutes at 100 μg/mL. Remaining photoresist was lifted off using varying concentrations of N-methyl-2-pyrrolidone (NMP, EMD Performance Materials) in MilliQ water. The devices were first submerged in a mixture of ⅔ MilliQ, ⅓ NMP for 20 seconds, then pure MilliQ water for 10 seconds. The devices were then submerged and sonicated in pure NMP for 6 minutes, then submerged and sonicated in a mixture of ½ MilliQ, ½ NMP for 1 minute. Finally, the devices were rinsed in fresh MilliQ water for 5 minutes, before we incubated the protein of interest on them. We used fluorescently-labeled gelatin (G13186, Thermofisher) to visualize the transferred protein patterns on the hydrogel devices, which we incubated on the devices for 60 minutes at room temperature. For devices that were seeded with hiPSC-CMs, we used Matrigel (356252, Corning) as the ECM protein at concentration of about 1000 μg/mL.

After protein incubation, polyacrylamide (PA) precursor solutions were prepared for casting the hydrogels using a previously published protocol with slight adjustments [35]. Briefly, we prepared 0.5 g/mL acrylamide (01696, Merck KGaA) and 0.025 g/mL bis-acrylamide (146072, Merck KGaA) solutions in MilliQ water. We combined 198 μL of the acrylamide solution and 40 of the bis-acrylamide solution, following the formulation for 10% T, 1% C hydrogels [35]. We added 21.6 μL of red fluorescent microbeads (F8812, Thermofisher), a necessary element for TFM analysis, along with 140.5 μL of 250 mM HEPES buffer (N-2-hydroxyethylpiperazine-N-2-ethane sulfonic acid, 15630080, Thermofisher). We adjusted the volume of MilliQ water to 594.4 μL, to account for the added volume of fluorescent microbeads and HEPES buffer. Separately, we prepared a 10% weight/volume solution of ammonium persulfate (APS, A9164, Merck KGaA) in MilliQ water. We degassed the PA precursor solutions and the APS solution in a vacuum desiccator for 1 hour.

To prepare for casting the hydrogels, 250 μm-thick polydimethylsiloxane (PDMS) spacers were introduced to define hydrogel thickness and make the hydrogel fabrication method compatible with different types of cell culture substrates when using 500 μm diced glass chips. Spacers are not needed when using coverslips due to the difference in weight.

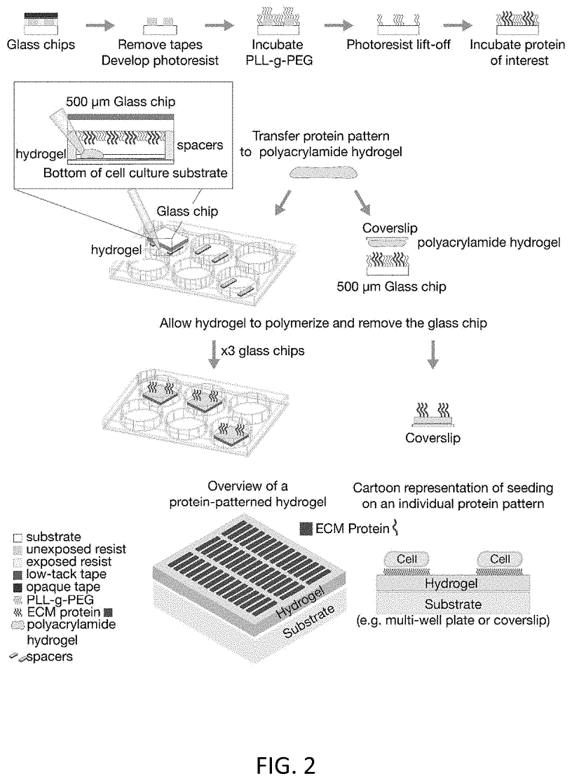

FIG. 2 outlines the process for fabricating hydrogels with diced glass chips and glass coverslips. For glass chips, PDMS spacers were placed in the well of a glass-bottom 6-well plate (P06-1.5H-N, Cellvis). The patterned glass chip was then placed on top of the PDMS spacers, with the patterned side of the glass facing downward.

To begin polymerization, 5 μL of the 10% APS solution and 0.5 μL of N,N,N′,N′-Tetramethylethylenediamine (TEMED, 411019, Merck KGaA) were added to the precursor solution. The solution was carefully mixed with a pipet, ensuring air bubbles were not introduced to the solution. For the diced glass chips, the solution was pipetted between the PDMS spacers until the solution spread throughout the entire sandwich, approximately 60 μL of solution total. For coverslips, 50 μL of the hydrogel solution was pipetted onto the cell culture substrate; then the coverslip was placed on top of the hydrogel solution, patterned side down.

Following casting, the hydrogels were protected from light and left for 30 minutes to begin polymerization. After 30 minutes, the hydrogels were hydrated with PBS and left to polymerize further at 4° C. for 6-8 hours. After full polymerization, the diced glass chips and coverslips were removed from the hydrogels and discarded.

It is important to note that hydrogel devices are not shelf stable [35] and should be stored in a buffer solution. We recommend that cells are seeded on devices within 72 hours of full polymerization.

### 2.4 Cardiomyocytes

In this work, we used human induced pluripotent stem cell-derived cardiomyocytes (hiPSC-CMs) (Coriell, AICS-0075-085) that were differentiated, expanded, and frozen using previously published protocols [36], [37].

Before seeding our devices, we thawed the hiPSC-CMs and cultured them in RPMI 1640 Medium with B27 supplement (B27, ThermoFisher) on a tissue culture 6-well plate coated with Matrigel Basement Membrane Matrix (Matrigel, Corning) for 10 days, changing the B27 every 2 days. After 10 days, we lifted up the hiPSC-CMs by incubating them in PBS+EDTA (ThermoFisher) for 5 to 10 minutes at 37° C. We then seeded these hiPSC-CMs on our hydrogel devices at a density of ˜20,000 cells/cm².

### 3. Results

3.1 Wafer fabrication and protein transfer to hydrogel devices

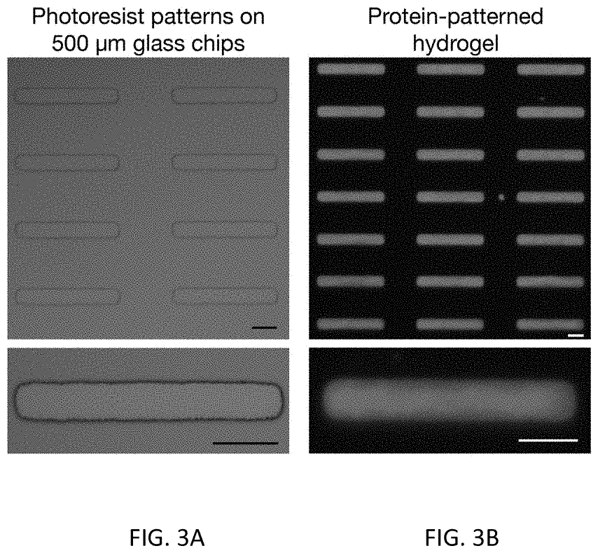

Wafer fabrication of photolithography patterns is a convenient and facile method for generating multiple chips to have on-demand for consistent hydrogel device fabrication. This method makes it easy to make multiple hydrogel devices with different properties in terms of stiffness or choice of extracellular matrix proteins, and the glass chips are compatible with different types of cell culture substrates, from glass coverslips to multi-well plates. FIG. 3 illustrates how the photoresist patterns (FIG. 3A) translate into protein patterns on a hydrogel (FIG. 3B) using fluorescently labeled gelatin.

To obtain high quality protein patterns on hydrogel devices, it is important to have photoresist patterns that are clean from debris and contaminations. For this reason, we develop photoresist post-wafer dicing, but it then becomes critical that the photoresist is minimally exposed to light at all steps of the fabrication process. We use masking tape during dicing to ensure protection from light exposure. This light sensitivity limited our ability to release UV tape on glass wafers using UV exposure and so using thicker glass wafers, 500 μm, significantly increased our yield of glass chips with photoresist patterns. However, thicker glass chips are also heavier than microscopy coverslips, and we introduced spacers in the hydrogel device fabrication method to define device thickness. This was critical to making the method compatible for different types of cell culture substrates when used with 500 μm glass chips. Without spacers, the order of the substrates during the hydrogel device fabrication matters and you become limited to using microscopy coverslips as substrates with dimensions that are smaller than those of the glass chips.

In developing the wafer fabrication process, we found that using glass wafers ultimately led to the highest quality transferred protein patterns in hydrogel devices. We tested wafers of glass, Si, and Si with 1 μm thermally grown oxide layer, however, using wafers other than glass could result in inverted protein patterns. These hydrogel devices were not used, and this was not a common occurrence when using glass wafers regardless of type or thickness. Si and Si with oxide layer were tested because the wafers are non-optically transparent and may be compatible with light-based release of UV tape after the dicing step.

### 4. Discussion

Our work shows that wafer scale lithography was successfully integrated with lift-off protein patterning and polyacrylamide hydrogels. In contrast, previous work utilized individual small coverslips during the lithography stage [34]. This serial fabrication ultimately results in a slow fabrication speed. In contrast, with our approach, many patterned photoresist coverslips can be made in parallel with one wafer. We decided to focus on streamlining the lithography stage with the intention to make lift-off patterning more accessible to other labs. The lithography stage requires cleanroom facilities and lithography expertise, which could be a significant access barrier. Our work could be used as a roadmap, to establish future collaboration with a cleanroom expertise team, in which wafer processing, lithography and dicing saw are common techniques. After the patterned photoresist coverslips are made, the rest of the protocol is straightforward and can be performed in a standard lab setup. Furthermore, our work shows that the diced and developed photoresist coverslips can be stored for at least six months. This is significant since it allows for streamlined batch processing and decreases the cleanroom time. This shelf-stable stage also allows for flexibility around cell culture maintenance.

Our work shows that lift-off patterning and the copolymerization transfer technique with polyacrylamide hydrogels is compatible with single cell hiPSC-CMs on Matrigel rectangular protein patterns. Previous work using lift-off protein patterning utilized Madin-Darby Canine Kidney (MDCK) cells on Collagen I and Gelatin protein patterns [34]. Furthermore, previous work also showed that lift-off is compatible with various hydrogel stiffness (5, 10, 25 kPa). Here, we only presented results with the polyacrylamide hydrogel formula for 10 kPa. This means that our approach can likely be expanded to other cell types, single cell or multiple cells, ECM protein types, protein pattern geometries, and hydrogel stiffness. Additionally, since this platform has live-cell microscopy compatibility other cell functional readouts can be easily added such as traction force microscopy [38]. Our approach presented here can be used in future studies to increase our understanding of mechanobiology and how the microenvironment influences cell structure and function in both healthy and disease states.

### REFERENCES

[1] “Underlying Cause of Death, 1999-2019 Request.” wonder.cdc.gov/ucd-icd10.html (accessed Aug. 25, 2021).
[2] S. S. Virani et al., “Heart Disease and Stroke Statistics—2020 Update: A Report From the American Heart Association,” Circulation, vol. 141, no. 9, pp. e139-e596, March 2020, doi: 10.1161/CIR.0000000000000757.
[3] B. J. Maron and M. S. Maron, “Hypertrophic cardiomyopathy,” The Lancet, vol. 381, no. 9862, pp. 242-255, January 2013, doi: 10.1016/S0140-6736(12)60397-3.
[4] A. J. Marian and E. Braunwald, “Hypertrophic Cardiomyopathy,” Circulation Research 121, Issue 7, 15 Sep. 2017; pp. 749-770.
[5] A. E. Becker and G. Caruso, “Myocardial disarray. A critical review,” Heart, vol. 47, no. 6, pp. 527-538, June 1982, doi: 10.1136/hrt.47.6.527.
[6] P. Garcia-Canadilla et al., “Myoarchitectural disarray of hypertrophic cardiomyopathy begins pre-birth,” Journal of Anatomy, vol. 235, no. 5, pp. 962-976, 2019, doi: 10.1111/joa.13058.
[7] R. C. Becker, A. P. Owens, and S. Sadayappan, “Tissue-Level Inflammation and Ventricular Remodeling in Hypertrophic Cardiomyopathy,” J Thromb Thrombolysis, vol. 49, no. 2, pp. 177-183, February 2020, doi:10.1007/s11239-019-02026-1.
[8] M. Chiong et al., “Cardiomyocyte death: mechanisms and translational implications,” Cell Death Dis, vol. 2, no. 12, p. e244, December 2011, doi: 10.1038/cddis.2011.130.
[9] M. T. BURROWS, “THE CULTIVATION OF TISSUES OF THE CHICK-EMBRYO OUTSIDE THE BODY,” Journal of the American Medical Association, vol. 55, no. 24, pp. 2057-2058, December 1910, doi:10.1001/jama.1910.04330240035009.
[10] A. R. Muir, “Further observations on the cellular structure of cardiac muscle,” J Anat, vol. 99, no. Pt 1, pp. 27-46, January 1965.
[11] V. Sander, G. Suñe, C. Jopling, C. Morera, and J. C. I. Belmonte, “Isolation and in vitro culture of primary cardiomyocytes from adult zebrafish hearts,” Nat Protoc, vol. 8, no. 4, pp. 800-809, April 2013, doi:10.1038/nprot.2013.041.
[12] M. Ackers-Johnson, P. Y. Li, A. P. Holmes, S.-M. O'Brien, D. Pavlovic, and R. S. Foo, “A Simplified, Langendorff-Free Method for Concomitant Isolation of Viable Cardiac Myocytes and Nonmyocytes From the Adult Mouse Heart,” Circ Res, vol. 119, no. 8, pp. 909-920, September 2016, doi: 10.1161/CIRCRESAHA.116.309202.
[13] N. Milani-Nejad and P. M. L. Janssen, “Small and Large Animal Models in Cardiac Contraction Research: Advantages and Disadvantages,” Pharmacol Ther, vol. 141, no. 3, pp. 235-249, March 2014, doi:10.1016/j.pharmthera.2013.10.007.
[14] R. Shephard and C. Semsarian, “Role of Animal Models in HCM Research,” J. of Cardiovasc. Trans. Res., vol. 2, no. 4, pp. 471-482, December 2009, doi: 10.1007/s12265-009-9120-y.
[15] P. Camacho, H. Fan, Z. Liu, and J.-Q. He, “Small mammalian animal models of heart disease,” Am J Cardiovasc Dis, vol. 6, no. 3, pp. 70-80, September 2016.
[16] E. Matsa, P. W. Burridge, and J. C. Wu, “Human stem cells for modeling heart disease and for drug discovery,” Sci Transl Med, vol. 6, no. 239, pp. 239ps6-239ps6, June 2014, doi: 10.1126/scitranslmed.3008921.
[17] J. Zhang et al., “Functional Cardiomyocytes Derived From Human Induced Pluripotent Stem Cells,” Circulation Research, vol. 104, no. 4, pp. e30-e41, February 2009, doi: 10.1161/CIRCRESAHA.108.192237.
[18] N. Sayed, C. Liu, and J. C. Wu, “Translation of Human iPSCs: From Clinical Trial in a Dish to Precision Medicine,” J Am Coll Cardiol, vol. 67, no. 18, pp. 2161-2176, May 2016, doi: 10.1016/j.j
[19] A. Schroer, G. Pardon, E. Castillo, C. Blair, and B. Prui, “Engineering hiPSC cardiomyocyte in vitro model systems for functional and structural assessment,” Progress in Biophysics and Molecular Biology, vol. 144, pp. 3-15, July 2019, doi: 10.1016/j.pbiomolbio.2018.12.001.
[20] I. Mannhardt et al., “Comparison of 10 Control hPSC Lines for Drug Screening in an Engineered Heart Tissue Format,” Stem Cell Reports, vol. 15, no. 4, pp. 983-998, October 2020, doi: 10.1016/j.stemcr.2020.09.002.
[21] X. Yang, L. Pabon, and C. E. Murry, “Engineering Adolescence: Maturation of Human Pluripotent Stem Cell-derived Cardiomyocytes,” Circ Res, vol. 114, no. 3, pp. 511-523, January 2014, doi:10.1161/CIRCRESAHA.114.300558.
[22] R. E. Ahmed, T. Anzai, N. Chanthra, and H. Uosaki, “A Brief Review of Current Maturation Methods for Human Induced Pluripotent Stem Cells-Derived Cardiomyocytes,” Frontiers in Cell and Developmental Biology, vol. 8, p. 178, 2020, doi: 10.3389/fce11.2020.00178.
[23] K. A. Jansen, D. M. Donato, H. E. Balcioglu, T. Schmidt, E. H. J. Danen, and G. H. Koenderink, “A guide to mechanobiology: Where biology and physics meet,” Biochim Biophys Acta, vol. 1853, no. 11 Pt B, pp. 3043-3052, November 2015, doi: 10.1016/j.bbamcr.2015.05.007.
[24] M. T. Lam and M. T. Longaker, “Comparison of several attachment methods for human iPS, embryonic and adipose-derived stem cells for tissue engineering,” Journal of Tissue Engineering and Regenerative Medicine, vol. 6, no. S3, pp. s80-s86, 2012, doi: 10.1002/term.1499.
[25] N. T. Kohen, L. E. Li le, and K. E. Healy, “Characterization of Matrigel interfaces during defined human embryonic stem cell culture,” Biointerphases, vol. 4, no. 4, pp. 69-79, December 2009, doi: 10.1116/1.3274061.
[26] C. S. Hughes, L. M. Postovit, and G. A. Lajoie, “Matrigel: A complex protein mixture required for optimal growth of cell culture,” PROTEOMICS, vol. 10, no. 9, pp. 1886-1890, 2010, doi:10.1002/pmic.200900758.
[27] T. Vignaud, H. Ennomani, and M. Théry, “Polyacrylamide hydrogel micropatterning,” Methods Cell Biol, vol. 120, pp. 93-116, 2014, doi: 10.1016/B978-0-12-417136-7.00006-9.
[28] S. Acevedo-Acevedo and W. C. Crone, “Substrate stiffness effect and chromosome missegregation in hIPS cells,” Journal of Negative Results in BioMedicine, vol. 14, no. 1, p. 22, December 2015, doi: 10.1186/s12952-015-0042-8.
[29] T. K. Borg, K. Rubin, E. Lundgren, K. Borg, and B. Obrink, “Recognition of extracellular matrix components by neonatal and adult cardiac myocytes,” Dev Biol, vol. 104, no. 1, pp. 86-96, July 1984, doi:10.1016/0012-1606(84)90038-1.
[30] S. Ba ista et al., “The effect of matrix composition of 3D constructs on embryonic stem cell differentiation,” Biomaterials, vol. 26, no. 31, pp. 6194-6207, November 2005, doi:10.1016/j.biomaterials.2005.04.003.
[31] L. L. Hilenski, X. H. Ma, N. Vinson, L. Terracio, and T. K. Borg, “The role of beta 1 integrin in spreading and myofibrillogenesis in neonatal rat cardiomyocytes in vitro,” Cell Motil Cytoskeleton, vol. 21, no. 2, pp. 87-100, 1992, doi: 10.1002/cm.970210202.
[32] M. L. Rodriguez, B. T. Graham, L. M. Pabon, S. J. Han, C. E. Murry, and N. J. Sniadecki, “Measuring the contractile forces of human induced pluripotent stem cell-derived cardiomyocytes with arrays of microposts,” J Biomech Eng, vol. 136, no. 5, p. 051005, May 2014, doi: 10.1115/1.4027145.
[33] A. J. S. Ribeiro et al., “Contractility of single cardiomyocytes differentiated from pluripotent stem cells depends on physiological shape and substrate stiffness,” Proc Natl Acad Sci USA, vol. 112, no. 41, p. 12705, October 2015, doi:10.1073/pnas.1508073112.
[34] J. Moeller, A. K. Denisin, J. Y. Sim, R. E. Wilson, A. J. S. Ribeiro, and B. L. Prui, “Controlling cell shape on hydrogels using lift-off protein patterning,” PLOS ONE, vol. 13, no. 1, p. e0189901, January 2018, doi:10.1371/journal.pone.0189901.
[35] A. K. Denisin and B. L. Prui, “Tuning the Range of Polyacrylamide Gel Stiffness for Mechanobiology Applications,” ACS Appl. Mater. Interfaces, vol. 8, no. 34, pp. 21893-21902, August 2016, doi: 10.1021/acsami.5b09344.
[36] A. Sharma, G. Li, K. Rajarajan, R. Hamaguchi, P. W. Burridge, and S. M. Wu, “Derivation of Highly Purified Cardiomyocytes from Human Induced Pluripotent Stem Cells Using Small Molecule-modulated Differentiation and Subsequent Glucose Starvation,” JoVE, no. 97, p. 52628, March 2015, doi: 10.3791/52628.
[37] J. W. Buikema et al., “Wnt Activation and Reduced Cell-Cell Contact Synergistically Induce Massive Expansion of Functional Human iPSC-Derived Cardiomyocytes,” Cell Stem Cell, vol. 27, no. 1, pp. 50-63.e5, July 2020, doi:10.1016/j.stem.2020.06.001.
[38] A. J. S. Ribeiro et al., “Contractility of single cardiomyocytes differentiated from pluripotent stem cells depends on physiological shape and substrate stiffness,” Proc Natl Acad Sci USA, vol. 112, no. 41, pp. 12705-12710, October 2015, doi: 10.1073/pnas.1508073112.

The above examples are provided to illustrate the invention but not to limit its scope. Other variants of the invention will be readily apparent to one of ordinary skill in the art and are encompassed by the appended claims. All publications, accessions, references, databases, and patents cited herein are hereby incorporated by reference for all purposes.

1. A method for performing protein patterning, the method comprising:
a) generating a photoresist pattern on a wafer using photolithography;
b) dicing the photoresist patterned wafer to obtain a plurality of photoresist patterned wafer chips; and
c) performing lift-off protein patterning on the plurality of photoresist patterned wafer chips to produce a protein pattern on the plurality of wafer chips.

2. The method of claim 1, further comprising transferring the protein pattern from the plurality of wafer chips to a hydrogel.

3. The method of claim 2, wherein said transferring comprises:
placing a protein patterned wafer chip on top of a spacer with the protein pattern facing downwards toward the spacer, wherein the spacer controls thickness of the hydrogel that forms;
adding a solution comprising a hydrogel polymer between the protein patterned wafer chip and the spacer;
polymerizing the hydrogel polymer to form the hydrogel, wherein the protein pattern is transferred from the protein patterned wafer chip to the hydrogel; and
removing the wafer chip from the hydrogel.

4. The method of claim 2, further comprising adding a cell to the hydrogel.

5. The method of claim 2, wherein the hydrogel has a stiffness ranging from about 1 kPa to 35 kPa.

6. The method claim 2, wherein the hydrogel comprises polyacrylamide.

7. The method of claim 1, wherein said performing lift-off protein patterning comprises:
incubating the plurality of photoresist patterned wafer chips with poly(l-lysine)-graft-poly (ethylene glycol) (PLL-g-PEG) copolymer;
removing the photoresist from the plurality of wafer chips;
and incubating the plurality of wafer chips with a protein of interest, wherein the protein of interest forms a pattern on the plurality of wafer chips.

8. The method of claim 7, wherein the photoresist is removed by submerging the wafer chips in an aqueous solution comprising N-methyl-2-pyrrolidone.

9. The method of claim 1, wherein the wafer has a thickness ranging from about 250 μm to about 700 μm.

10. The method of claim 9, wherein the wafer has a thickness of about 500 μm.

11. The method claim 1, wherein the wafer has a diameter ranging from about 50 mm to about 300 mm.

12. The method of claim 11, wherein the wafer has a diameter of about 100 mm.

13. The method claim 1, wherein the wafer is a glass wafer or a silicon wafer.

14. The method of claim 13, wherein the silicon wafer further comprises an oxide layer.

15. The method claim 1, further comprising protecting the photoresist pattern from light exposure during said dicing by covering the wafer with an opaque material.

16. The method of claim 15, wherein the opaque material is an opaque tape.

17. The method of claim 1, wherein the wafer is diced with a dicing saw.

18. The method of claim 17, wherein the dicing saw comprises a thermocarbon diamond blade.

19. The method of claim 1, wherein said dicing produces at least 16 chips per wafer.

20. The method of claim 1, wherein the wafer chips are square or rectangular.

21. The method of claim 20, wherein the square has a side having a length of about 15 mm.

22. The method of claim 1, wherein said lift-off protein patterning is performed with one or more extracellular matrix proteins.

23. The method of claim 22, wherein the one or more extracellular matrix proteins are laminin, fibronectin, collagen, elastin, nidogen, heparan sulfate, chondroitin sulfate, keratan sulfate, or hyaluronic acid, or any combination thereof.

24. The method of claim 1, further comprising storing the photoresist patterned wafer or the photoresist patterned wafer chips for a period of time prior to said performing lift-off protein patterning.
